(19) United States
(12) Patent Application Publication
(10) Pub. No.: US 2005/0118139 A1
(43) Pub. Date: Jun. 2, 2005

(54) Vaccine using papilloma virus e proteins delivered by viral vector

(76) Inventors: Lingyi Huang, Norristown, PA (US); Kathrin U. Jansen, Doylestown, PA (US); William L. McClements, Doylestown, PA (US); Juanita M. Monteiro, Cheltenham, PA (US); Loren D. Schultz, Harleysville, PA (US); Timothy W`. Tobery, Wilmington, DE (US); Xin-Min Wang, Schwenksville, PA (US); Ling Chen, The Woodlands, TX (US)

Correspondence Address: MERCK AND CO., INC, P O BOX 2000, RAHWAY, NJ, 07065-0907, US

(21) Appl. No.: 10/487,148

(22) PCT Filed: Aug. 19, 2002
(86) PCT No.: PCT/US02/26965

Related U.S. Application Data
(60) Provisional application No. 60/314,395, filed on Aug. 23, 2001.

(51) Int. Cl. A61K 48/00; C12N 15/861

(52) U.S. Cl. 424/93.2; 514/44; 435/456

(57) ABSTRACT

Cell-mediated immune response to a papillomavirus infection can be induced by vaccination with DNA encoding papillomavirus E genes. E genes can both prevent the occurrence of papillomavirus disease, and treat disease states. Canine papillomavirus (COPV) E genes which are codon-optimized to enhance expression in host cells are also given.

### BRIEF DESCRIPTION OF THE INVENTION

This invention relates to a vaccine inducing cell-mediated immunity which comprises a vector encoding a papillomavirus E gene, and the prevention and/or treatment of disease caused by the papillomavirus. This invention also relates to adenoviral vector constructs carrying canine papillomavirus (COPV) “E” genes, and to their use as vaccines. Further inventions also relates to various COPV genes which have been codon-optimized, and to methods of using the adenoviral constructs.

### BACKGROUND OF THE INVENTION

Papillomavirus infections occur in a variety of animals, including humans, sheep, dogs, cats, rabbits, snakes, monkeys and cows. Papillomaviruses infect epithelial cells, generally inducing benign epithelial or fibroepithelial tumors at the site of infection. Papillomaviruses are species specific infective agents; a human papillomavirus cannot infect a non-human.

Papillomaviruses are small (50-60 nm), nonenveloped, icosahedral DNA viruses what encode up to eight early and two late genes. The open reading frames (ORFs) of the virus are designated E1 to E7 and L1 and L2, where “E” denotes early and “L” denotes late. L1 and L2 code for virus capsid proteins. The early genes are associated with functions such as viral replication and cellular transformation.

In humans, different HPV types cause distinct diseases, ranging from benign warts (for examples HPV types 1, 2, 3) to highly invasive genital and anal carcinomas (HPV types 16 and 18). At present there is not a satisfactory therapeutic regimen for these diseases.

In dogs, canine oral papilloma virus (COPV) causes a transitory outbreak of warts in the mouth. In rabbits, cottontail rabbit papilloma virus (CRPV) can cause cornified warty growths on the skin.

Immunological studies in animals (including dogs) have shown that the production of neutralizing antibodies to papillomavirus antigens prevents infection with the homologous virus. Furthermore, immunization of dogs with DNA encoding the L1 capsid protein of COPV induces neutralizing antibodies and protects dogs from COPV-induced disease. In rabbits, immunization with DNA encoding CRPV L1 induces neutralizing antibodies that are partially protective against CRPV disease. Also it has been shown that immunization with DNA encoding CRPV E proteins, can also partially protect domestic rabbits from the development of warts in the absence of neutralizing antibodies. [Han, R. et al. 1999a J Virol 73(8), 7039-43; Han, R. et al 1999b Vaccine 17(11-12), 1558-66; Sundaram, P. et al 1997 Vaccine 15(6-7), 664-71; Sundaram, P., et al, 1998. Vaccine 16(6), 613-23.]

### SUMMARY OF THE INVENTION

This invention relates to the induction of cell-mediated immune responses by immunization of animals with adenovirus vectors carrying genes which encode papillomavirus E proteins (regardless of viral type), and to the protection of immunized animals from disease. The disease can be induced by infection with a papillomavirus or it can be a model disease such as protection from tumor outgrowth by cells expressing an E protein as a model tumor antigen.

Thus, this invention relates to a method of preventing a disease caused by a papillomavirus comprising the steps of administering to a mammal a vaccine vector comprising a papillomavirus E gene. This invention also relates to a method of treating a disease caused by a papillomavirus comprising administering to a mammal exhibiting symptoms of the disease a vector comprising a papillomavirus E gene. In both of these inventions, the mammal is preferably a human, and the vector may be either an adenovirus vector or a plasmid vector, and the genes are preferably from a human papillomavirus (HPV) serotype which is associated with a human disease state. The disease may be, for example, cervical carcinoma, genital warts, or any other disease which is associated with a papillomavirus infection.

In some embodiments of this invention, protection from disease, or alternatively treatment of existing disease is induced by immunization with vectors encoding a protein selected from the group consisting of: E1, E2, E4, E5, E6 and E7 proteins, and combinations thereof. The E proteins which are particularly preferred are E1 and E2 proteins, delivered either separately or in combination. The polynucleotide encoding the E protein is preferable codon-optimized for expression in the recipient's cells.

In a particularly preferred embodiment, the vector is an adenoviral vector comprising an adenoviral genome with a deletion in the adenovirus E1 region, and an insert in the adenovirus E1 region, wherein the insert comprises an expression cassette comprising:
a) a polynucleotide encoding a papillomavirus protein selected from the group consisting of E1, E2, E4, E5, E6, E7, and combinations thereof, or mutant forms thereof, wherein the polynucleotide is codon-optimized for expression in a human host cell; and
b) a promoter operably linked to the polynucleotide. The preferred adenovirus may be an Ad 5 adenovirus, but other serotypes may be used, particularly if one is concerned about interaction between the adenoviral vector and the patients' preexisting antibodies.

Another type of vector which is envisioned by this invention is a shuttle plasmid vector comprising a plasmid portion and an adenoviral portion, the adenoviral portion comprising: an adenoviral genome with a deletion in the adenovirus E1 region, and an insert in the adenovirus E1 region, wherein the insert comprises an expression cassette comprising:
a) a polynucleotide encoding an E protein selected from the group consisting of—E1, E2, E4, E5, E6, E7, and combinations thereof, or mutant forms thereof, wherein the polynucleotide is codon-optimized for expression in a mammalian host cell; and
b) a promoter operably linked to the polynucleotide.

This invention also is directed to plasmid vaccine vectors, which comprise a plasmid portion and an expressible cassette comprising
a) a polynucleotide encoding an E protein selected from the group consisting of E1, E2, E4, E5, E6, E7 and combinations thereof, or mutant forms thereof, wherein the polynucleotide is codon-optimized for expression in a mammalian host cell; and
b) a promoter operably linked to the polynucleotide.

Yet another aspect of this invention are host cells containing these vectors.

This invention also relates to oligonucleotides which encode a canine oral papillomavirus (COPV) protein which have been codon-optimized for efficient expression in a host cell; preferably the oligonucleotides are DNA.

This invention also relates to a method of making a COPV E protein comprising expressing in a host cell a synthetic polynucleotide encoding a COPV E protein, or mutated form of the COPV E protein which has reduced protein function as compared to wild-type protein, but which maintains immunogenicity, the polynucleotide sequence comprising codons optimized for expression in a mammalian host.

### BRIEF DESCRIPTION OF THE FIGURES

FIG. 1 is the nucleotide sequence of a codon-optimized COPV E1 gene (SEQ.ID.NO:1).

FIG. 2 is the nucleotide sequence of a codon-optimized COPV E2 gene (SEQ.ID.NO:2).

FIG. 3 is the nucleotide sequence of a codon-optimized COPV E4 gene (SEQ.ID.NO:3).

FIG. 4 is the nucleotide sequence of a codon-optimized COPV E7 gene SEQ.ID.NO:4). In this particular sequence, the cysteine residue at position 24 has been changed to glycine, and the glutamic acid residue at position 26 has been changed to a glycine.

FIG. 5 is a table showing cell-mediated immune responses in mice immunized with either an E protein or an L protein.

FIG. 6 is a graph showing the protection of mice from HPV E2 tumor challenge by immunization with Ad-TO-HPV16E2.

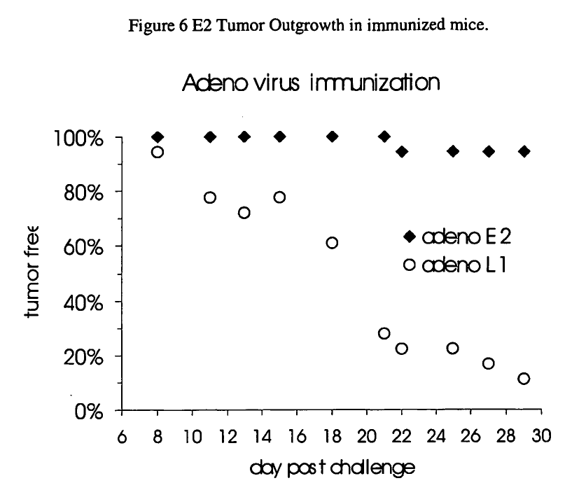

FIG. 7 is a table showing specific cellular immune response in Rhesus macaques following immunization with Ad5-HPV16 constructs

FIG. 8 is a table summarizing the results of immunizing beagles with Ad-COPV E vaccines.

### SUMMARY OF THE INVENTION

The term “promoter” as used herein refers to a recognition site on a DNA strand to which the RNA polymerase binds. The promoter forms an initiation complex with RNA polymerase to initiate and drive transcriptional activity. The complex can be modified by activating sequences termed “enhancers” or inhibiting sequences termed “silencers”.

The term “cassette” refers to the sequence of the present invention which contains the nucleic acid sequence which is to be expressed. The cassette is similar in concept to a cassette tape; each cassette has its own sequence. Thus by interchanging the cassette, the vector will express a different sequence. Because of the restrictions sites at the 5′ and 3′ ends, the cassette can be easily inserted, removed or replaced with another cassette.

The term “vector” refers to some means by which DNA fragments can be introduced into a host organism or host tissue. There are various types of vectors including plasmid, virus (including adenovirus), bacteriophages and cosmids.

The term “effective amount” means sufficient vaccine composition is introduced to produce the adequate levels of the polypeptide, so that an immune response results. One skilled in the art recognizes that this level may vary.

“Synthetic” means that the COPV gene has been modified so that it contains codons which are preferred for mammalian expression. In many cases, the amino acids encoded by the gene remain the same. In some embodiments, the synthetic gene may encode a modified protein.

“Mutant” as used throughout this specification and claims requires that if referring to a nucleic acid, the protein encoded has at least the same type of biological function as the wild-type protein, although the mutant may have an enhanced or diminished function; or if referring to a protein, the mutant protein has at least the same type of biological function as the wild-type protein, although the mutant may have an enhanced or diminished function.

The term “native” means that the gene contains the DNA sequence as found in occurring in nature. It is a wild type sequence of viral origin.

### DETAILED DESCRIPTION OF THE INVENTION

Synthetic DNA molecules encoding various HPV proteins and COPV proteins are provided. The codons of the synthetic molecules are designed so as to use the codons preferred by the projected host cell, which in preferred embodiments is a human cell. The synthetic molecules may be used in a recombinant adenovirus vaccine which provides effective immunoprophylaxis against papillomavirus infection through cell-mediated immunity.

The recombinant adenovirus vaccine may also be used in various prime/boost combinations with a plasmid-based polynucleotide vaccine. This invention provides polynucleotides that, when directly introduced into a vertebrate in vivo, including mammals such as primates, dogs and humans, induce the expression of encoded proteins within the animal.

The vaccine formulation of this invention may contain a mixture of recombinant adenoviruses encoding different HPV type protein genes (for example, genes from HPV6, 11, 16 and 18), and/or it may also contain a mixture of protein genes (i.e. L1, E1, E2, E4 and/or E7). In similar fashion, the vaccine formulation of this invention may contain a mixture of recombinant adenoviruses, each encoding different a different papillomavirus protein gene (for example, L1, E1, E2, E4 and/or E7). E2 genes are particularly preferred.

Serotypes of HPV which are useful in the practice of this invention include: HPV6a, HPV6b, HPV11, HPV16, HPV18, HPV31, HPV33, HPV35, HPV39, HPV45, HPV51, HPV52, HPV56, HPV58, and HPV68.

Codon Optimization

The wild-type sequences for HPV and COPV genes are known. In accordance with this invention, papillomavirus gene segments were converted to sequences having identical translated amino acid sequences but with alternative codon usage as defined by Lathe, 1985 “Synthetic Oligonucleotide Probes Deduced from Amino Acid Sequence Data: Theoretical and Practical Considerations” J. Molec. Biol. 183:1-12, which is hereby incorporated by reference. The methodology may be summarized as follows:
1. Identify placement of codons for proper open reading frame.
2. Compare wild type codon for observed frequency of use by human genes.
3. If codon is not the most commonly employed, replace it with an optimal codon for high expression in human cells.
4. Repeat this procedure until the entire gene segment has been replaced.
5. Inspect new gene sequence for undesired sequences generated by these codon replacements (e.g., “ATTTA” sequences, inadvertent creation of intron splice recognition sites, unwanted restriction enzyme sites, etc.) and substitute codons that eliminate these sequences.
6. Assemble synthetic gene segments and test for high-level expression in mammalian cells.

These methods were used to create the following synthetic gene segments for various papillomavirus genes by creating a gene comprised entirely of codons optimized for high level expression. While the above procedure provides a summary of our methodology for designing codon-optimized genes for DNA vaccines, it is understood by one skilled in the art that similar vaccine efficacy or increased expression of genes may be achieved by minor variations in the procedure or by minor variations in the sequence.

In some embodiments of this invention, alterations have been made (particularly in the E-protein native protein sequences) to reduce or eliminate protein function while preserving immunogenicity. Mutations which decrease enzymatic function are known. Certain alterations were made for purposes of expanding safety margins and/or improving expression yield. These modifications are accomplished by a change in the codon selected to one that is more highly expressed in mammalian cells.

In accordance with this invention, COPV E7, conversion of cysteine at position 24 to glycine and glutamic acid at position 26 to glycine was permitted by alteration of TGC and the GAG to GGA and GGC, respectively. For HPV, mutants include HPV 16 E1 where glycine at amino acid 482 is changed to aspartic acid and tryptophan at 439 is changed to arginine. For HPV16 E2, a mutant changes glutamic acid at position 39 to alanine; for HPV 16 E7, a mutant changes cysteine at position 24 to glycine, and glutamic acid at 26 is changed to glycine.

The codon-optimized genes are then assembled into an expression cassette which comprises sequences designed to provide for efficient expression of the protein in a human cell. The cassette preferably contains the codon-optimized gene, with related transcriptional and translations control sequences operatively linked to it, such as a promoter, and termination sequences. In a preferred embodiment, the promoter is the cytomegalovirus promoter with the intron A sequence (CMV-intA), although those skilled in the art will recognize that any of a number of other known promoters such as the strong immunoglobulin, or other eukaryotic gene promoters may be used. A preferred transcriptional terminator is the bovine growth hormone terminator, although other known transcriptional terminators may also be used. The combination of CMVintA-BGH terminator is particularly preferred.

Examples of preferred gene sequences for COPV E1, E2, E4 and mutant E7 (C24G, E26G) are given in SEQ.ID.NOS: 1-4.

Vectors

In accordance with this invention, the expression cassette encoding at least one papillomavirus protein is then inserted into a vector. The vector is preferably an adenoviral vector, although linear DNA linked to a promoter, or other vectors, such as adeno-associated virus or a modified vaccinia virus vector may also be used.

If the vector chosen is an adenovirus, it is preferred that the vector be a so-called first-generation adenoviral vector. These adenoviral vectors are characterized by having a non-functional E1 gene region, and preferably a deleted adenoviral E1 gene region. In some embodiments, the expression cassette is inserted in the position where the adenoviral E1 gene is normally located. In addition, these vectors optionally have a non-functional or deleted E3 region. The adenoviruses can be multiplied in known cell lines which express the viral E1 gene, such as 293 cells, or PERC.6 cells, or in cell lines derived from 293 or PERC.6 cell which are transiently or stablily transformed to express an extra protein. For examples, when using constructs that have a controlled gene expression, such as a tetracycline regulatable promoter system, the cell line may express components involved in the regulatory system. One example of such a cell line is T-Rex-293; others are known in the art.

For convenience in manipulating the adenoviral vector, the adenovirus may be in a shuttle plasmid form. This invention is also directed to a shuttle plasmid vector which comprises a plasmid portion and an adenovirus portion, the adenovirus portion comprising an adenoviral genome which has a deleted E1 and optional E3 deletion, and has an inserted expression cassette comprising at least one codon-optimized papillomavirus gene. In preferred embodiments, there is a restriction site flanking the adenoviral portion of the plasmid so that the adenoviral vector can easily be removed. The shuttle plasmid may be replicated in prokaryotic cells or eukaryotic cells.

Standard techniques of molecular biology for preparing and purifying DNA constructs enable the preparation of the adenoviruses, shuttle plasmids and DNA immunogens of this invention.

In some embodiment of this invention, both the adenoviral vectors vaccine and a plasmid vaccine may be administered to a vertebrate in order to induce an immune response. In this case, the two vectors are administered in a “prime and boost” regimen. For example the first type of vector is administered, then after a predetermined amount of time, for example, 1 month, 2 months, six months, or other appropriate interval, a second type of vector is administered. Preferably the vectors carry expression cassettes encoding the same polynucleotide or combination of polynucleotides. In the embodiment where a plasmid DNA is also used, it is preferred that the vector contain one or more promoters recognized by mammalian or insect cells. In a preferred embodiment, the plasmid would contain a strong promoter such as, but not limited to, the CMV promoter. The gene to be expressed would be linked to such a promoter. An example of such a plasmid would be the mammalian expression plasmid V1Jns as described (J. Shiver et. al. 1996, in DNA Vaccines, eds., M. Liu, et al. N.Y. Acad. Sci., N.Y., 772:198-208 and is herein incorporated by reference).

Thus, another aspect of this invention is a method for inducing an immune response against a papillomavirus in a mammal, comprising
A) introducing into the mammal a first vector comprising a polynucleotide encoding a papillomavirus protein selected from the groups consisting of E1, E2, E4, E6, E7, combinations thereof, and mutants thereof;
B) allowing a predetermined amount of time to pass;
C) introducing into the mammal a second vector comprising an adenoviral genome with a deletion in the E1 region, and an insert in the E1 region, wherein the insert comprises an expression cassette comprising:
i) a polynucleotide encoding an COPV protein selected from the group consisting of, E1, E2, E4, and E7 proteins, combinations thereof, and mutant forms thereof; and
ii) a promoter operably linked to the polynucleotide.

In some embodiments, the first vector be a plasmid vaccine vector and the second vector be an adenoviral vector.

In yet another embodiment of this invention, the codon-optimized genes are introduced into the recipient by way of a plasmid or adenoviral vector, as a “priming dose”, and then a “boost” is accomplished by introducing into the recipient a polypeptide or protein which is essentially the same as that which is encoded by the codon-optimized gene. Fragments of a full length protein may be substituted, especially those with are immunogenic and/or include an epitope.

It is also a part of this invention to combine the use of the nucleotide based vaccines with the administration of a protein. The protein may be an L1 protein, or an L1 in combination with an L2 protein. It is particularly preferred that the protein be in the form of a VLP. The VLP may be a human papillomavirus VLP. Such VLPs are known and described in the art.

The amount of expressible DNA or transcribed RNA to be introduced into a vaccine recipient will depend partially on the strength of the promoters used and on the immunogenicity of the expressed gene product. In general, an immunologically or prophylactically effective dose of about 1 ng to 100 mg, and preferably about 10%1 g to 300 μg of a plasmid vaccine vector is administered directly into muscle tissue. An effective dose for recombinant adenovirus is approximately 10⁶-10¹² particles and preferably about 10⁷-10¹¹ particles. Subcutaneous injection, intradermal introduction, impression though the skin, and other modes of administration such as intraperitoneal, intravenous, or inhalation delivery are also contemplated. It is also contemplated that booster vaccinations may be provided. Parentaeral administration, such as intravenous, intramuscular, subcutaneous or other means of administration with adjuvants such as interleukin 12 protein, concurrently with or subsequent to parenteral introduction of the vaccine of this invention is also advantageous.

The vaccine vectors of this invention may be naked, i.e., unassociated with any proteins, adjuvants or other agents which impact on the recipient's immune system. In this case, it is desirable for the vaccine vectors to be in a physiologically acceptable solution, such as, but not limited to, sterile saline or sterile buffered saline. Alternatively, the DNA may be associated with an adjuvant known in the art to boost immune responses, such as a protein or other carrier. Agents which assist in the cellular uptake of DNA, such as, but not limited to calcium ion, may also be used to advantage. These agents are generally referred to as transfection facilitating reagents and pharmaceutically acceptable carriers.

The following examples are offered by way of illustration and are not intended to limit the invention in any manner.

### EXAMPLE 1

Synthetic Gene Construction

The construction of synthetic codon-optimized gene sequences for human papillomavirus type 16 proteins L1, E1, and E2 was disclosed previously (International Publication Number WO 01/14416A2, publication date: 1 Mar. 2001, “Synthetic Human Papillomavirus Genes” which is hereby incorporated by reference). Synthetic gene sequences for canine oral papillomavirus proteins E1, E2, and E7 were generated by reverse translation of amino acid sequences using the most frequently used codons found in highly expressed mammalian genes. (R. Lathe, 1985, J. Mol. Biol. 183:1-12, which is hereby incorporated by reference). Some adjustments to these codon-optimized sequences were made to introduce or remove restriction sites.

Oligonucleotides based on these sequences were chemically synthesized (Midland Certified Reagents; Midland, Tex.) and assembled by PCR amplification. (J. Haas et. al., 1996, Current Biology 6:315-324; and PCR Protocols, M. Innis, et al, eds., Academic Press, 1990, both of which are hereby incorporated by reference).

Full-length sequences were cloned into the mammalian expression vector V1Jns (J. Shiver et. al. 1996, in DNA Vaccines, eds., M. Liu, et al. N.Y. Acad. Sci., N.Y., 772:198-208, which is hereby incorporated by reference) and sequenced by standard methodology. In cases where the actual sequence differed from the expected and resulted in amino acid substitution, that sequence was corrected by PCR mutagenesis as previously described (PCR Protocols, M. Innis, et al, eds., Academic Press, 1990, pg 177-180).

Protein expression was evaluated by transient transfection of equal quantities of plasmid DNA into 293 (transformed embryonic human kidney) cells or C33a cells which were harvested at 48 hr post DNA addition. Cell lysates were normalized to provide equal protein loadings. Analysis was by immunoblot (Western) analysis using sera prepared to each of the COPV proteins. (Current Protocols in Molecular Biology, eds., F. Ausabel, et. Al., John Wiley and Sons, 1998, which is hereby incorporated by reference).

### EXAMPLE 2

Synthesis of COPV E1

The gene encoding COPV E1 was prepared by the annealing and extension of 24 oligomers (83-108 bp in length) designed to encode the final desired sequence. The oligomers were alternating, overlapping sense and antisense sequences which spanned the entire length of the optimized COPV E1 coding sequence as well as providing the following important sequence elements: (1) BglII and EcoRV restriction sites plus a CCACC “Kozak sequence” upstream of the ATG initiation codon and (2) EcoRV and BglII restriction sites downstream of the translation termination codon at the extreme 5′ and 3′ ends of the synthetic full-length sequence. Each oligomer had a complementary overlap region of 23-27 bp with the adjoining oligomer (duplex had Tm of 78-86° C.). Six separate extension reactions were performed using four adjoining, overlapping oligomers and sense and antisense PCR primers (20-25 nt in length, Tm=68-70° C.) complementary to the distal 5′ and 3′ portions of the first and fourth oligomer, respectively. The actual conditions of PCR were similar to those described in EXAMPLES 3 and 4 of International Publication Number WO 01/14416A2.

As a result of these PCR reactions, the following six fragments of the gene were created: COPV E1-A, COPV E1-B, COPV E1-C, COPV E1-D, COPV E1-E and COPV E1-F.

The above fragments resulting from the PCR reactions were gel separated on low melting point agarose with the appropriately-sized products excised and purified using the Agarase™ method (Boehringer Mannheim Biochemicals) as recommended by the manufacturer. Fragments COPV E1-A, COPV E1-B and COPV E1-C were combined in a subsequent PCR reaction using appropriate distal sense and antisense PCR oligomers as described previously (International Publication Number WO 01/14416A2), yielding the PCR product COPV E1-G. In a similar manner, fragments COPV E1-D, COPV E1-E and COPV E1-F were assembled in a subsequent PCR reaction with the appropriate primers to yield the fragment COPV E1-H. The complete gene was then assembled by an additional PCR reaction in which fragments COPV E1-G and COPV E1-H were combined using appropriate distal sense and antisense PCR primers. The resulting 1.8 kb product (designated COPV E1-I) was gel isolated, digested with Bgl II and subcloned into the expression vector V1Jns and a number of independent isolates were sequenced. In instances where a mutation was observed, it was corrected by assembling overlapping portions of COPV E1 gene segments from different isolates that had the correct sequence.

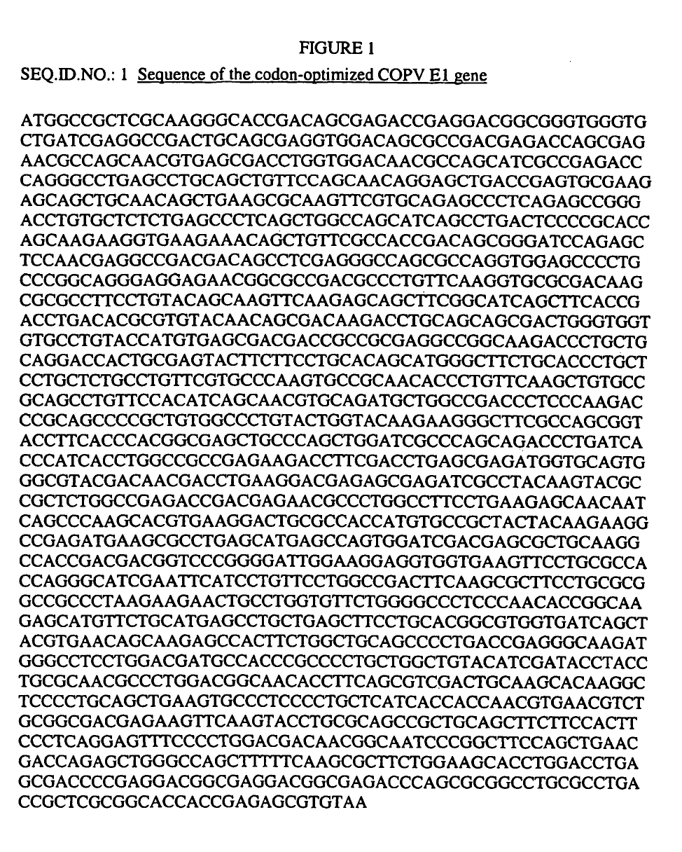

Standard PCR methods as described above were used. DNA was isolated from a final clone with the correct COPV E1 DNA sequence and proper orientation within V1Jns for use in transient transfection assays as described in EXAMPLE 1. The sequence of the codon-optimized ORF for COPV E1 is shown in FIG. 1 (SEQ.ID.NO.:1).

Immunoblot analyses of cell lysates prepared from the transfected cells verified the expression of a protein of the expected size which reacted with antibodies directed against COPV E1 (results not shown).

### EXAMPLE 3

Synthesis of COPV E2, COPV E4 and COPV E7 Genes

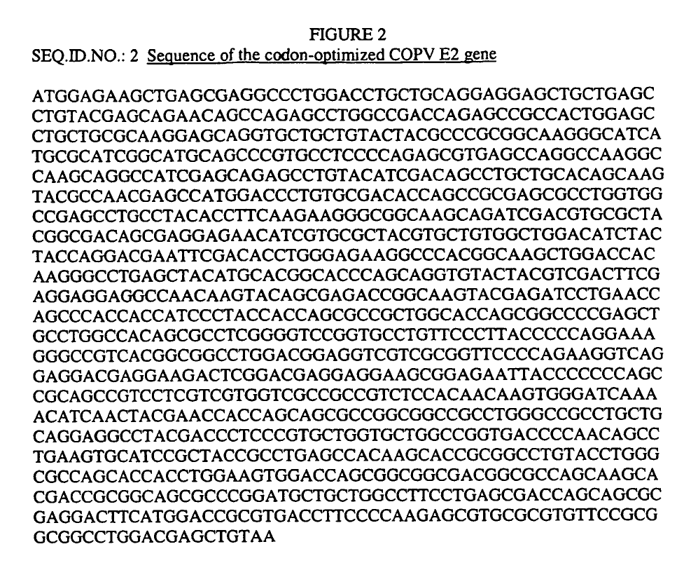

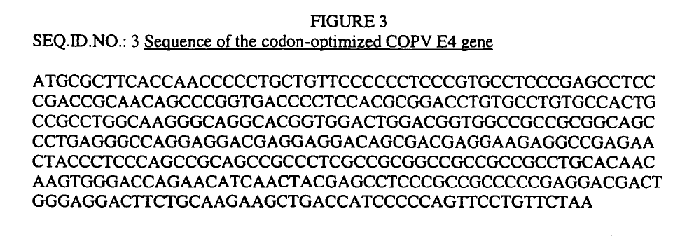

The synthetic genes encoding the codon-optimized versions of the COPV E2, COPV°E4 and COPV E7 proteins were prepared using the same type of construction strategy using annealing and extension of long DNA oligomers as described in Example 2 and in International Publication Number WO 01/14416A2. The sequences used for the long DNA oligomers and PCR primers used for assembly of the oligomers and resulting gene fragments were designed according to the criteria in Example 2 in order to give the following final coding sequences: COPV E2, FIG. 2 (SEQ.ID.NO.:2); COPV E4, FIG. 3 (SEQ.ID.NO.:3).

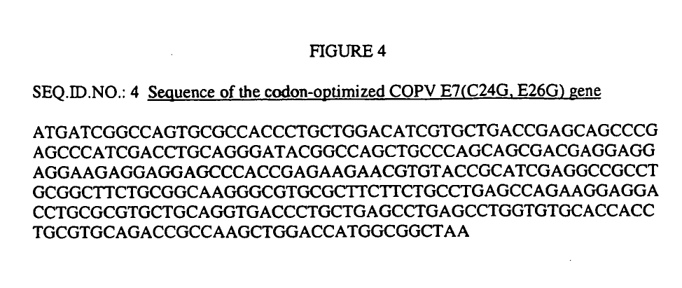

The codon-optimized COPV E7 gene was initially constructed to encode the wild-type COPV E7 protein sequence. The double mutant (C24G, E26G) version of COPV E7 was prepared by PCR mutagenesis by converting TGC at codon 24 to GGA and by converting GAG at codon 26 to GGC. The methods for the PCR mutagenesis were as previously described (PCR Protocols, M. Innis, et al, eds., Academic Press, 1990, pg 177-180). The final coding sequence used for COPV E7 (C24G,E26G) is shown in FIG. 4 (SEQ.ID.NO.:4).

For all three of these synthetic genes, the following sequence elements were also present in the final assembled gene fragment in addition to the protein coding sequence: (1) BglII and PmlI restriction sites plus a CCACC “Kozak sequence” upstream of the ATG initiation codon and (2) PmlI and BglII restriction sites downstream of the translation termination codon. As described above for COPV E1, each of the three gene fragments was digested with BglII and cloned into the expression vector V1Jns. Following verification of the DNA sequences, purified plasmid DNAs for each of the three constructs were used for transient transfection assays as described in Example 1.

For COPV E2, COPV E4 and COPV E7, immunoblot analyses of cell lysates prepared from the cells transfected with the corresponding vector verified the expression of a protein of the expected size which reacted with antibodies directed against that particular COPV protein (results not shown).

### EXAMPLE 4

Construction of Replication-Defective Adenovirus Expressing HPV or COPV Antigens

Shuttle vector pHCMVIBGHpA1 contains Ad5 sequences from bp1 to bp 341 and bp 3534 to bp 5798 with a expression cassette containing human cytomegalovirus (HCMV) promoter plus intron A and bovine growth hormone polyadenylation signal.

The adenoviral backbone vector pAdE1-E3—(also named as pHVad1) contains all Ad5 sequences except those nucleotides encompassing the E1 and E3 region.

Construction of Ad5-HPV16E1: The HPV16 E1 coding sequence was excised from V1Jns-HPV16E1 by digestion with BglII and cloned into the BglII site located between the CMV promoter and BGH terminator in pHCMVIBGHpA1. The resulting shuttle vector was recombined with the adenovirus backbone vector DNA as described previously (International Publication Number WO 01/14416A2). The resulting recombinant virus, Ad5-HPV16E1, was then isolated and amplified in 293 cells as described in that same reference.

Construction of Ad5-TO-HPV16L1:

Construction of Adenoviral Shuttle Plasmid pA1-TO-HPV16L1 Containing HPV16L1 Under Control of the Regulated CMV-TO Promoter.

The construction of the plasmid HPV16L1/V1Jns, which contains the codon-optimized synthetic coding sequence for HPV16L1 was described previously (International Publication Number WO 01/14416A2, publication date: 1 Mar. 2001, Synthetic Human Papillomavirus Genes). The synthetic HPV16L1 coding sequence was excised from HPV16L1/V1Jns by digestion with BglII plus EcoRI and then cloned into BglII, EcoRI-digested pHCMVIBGHpA1 to yield the shuttle vector pA1-CMVI-HPV16L1. The shuttle vector pA1-CMVI-HPV16L1 was digested with BglII plus SpeI (to remove the CMV promoter plus intron A sequences), made flushended and the large vector fragment was gel-purified.

The mammalian expression vector pcDNA4/TO (Invitrogen Corp.) contains two copies of the tetracycline operator (TetO₂) sequence inserted 10 bp downstream of the TATA box sequence for the human CMV promoter present in that vector. Presence of the tetracycline operator (TetO₂) sequence results in repression of expression in host cells that express the Tetracycline repressor. The pcDNA4/TO vector was digested with NruI plus EcoRV and the 823 bp fragment bearing the CMV promoter plus tetracycline operator (2×TetO₂) sequences (CMV-TO) was gel-purified and ligated with the aforementioned 8.3 kbp BglII-SpeI (flushended) fragment bearing the HPV16L1 coding sequence. The resulting plasmid was designated pA1-TO-HPV16L1.

Homoloogus Recombination to Generate Shuttle Plasmid Form of Recombinant Adenoviral Vector pAd-TO-HPV16L1.

Shuttle plasmid pA1-TO-HPV16L1 was digested with restriction enzymes SspI and BstZ17I and then co-transformed into E. coli strain BJ5183 with linearized (ClaI-digested) adenoviral backbone plasmid pAdE1-E3-. Eight colonies were picked from the resulting transformation plate and separately grown in 2-ml of Terrific Broth containing 50 mcg/ml of ampicillin. Small-scale plasmid DNA preparation were made and then used for transformation of E. coli STBL2 competent cells (Life Technologies). From each of the resulting transformation plates, a single colony was picked and inoculated into LB with ampicillin (50 mcg/ml) and grown overnight at 37° C. Plasmid DNA was prepared from each culture and restriction enzyme analysis was used to verify that the pAd5-TO-HPV16L1 plasmids had the correct structure.

Generation of Recombinant Adenovirus Ad5-TO-HPV16L1 in T-REx-293 cells

The shuttle plasmid pAd-TO-HPV16L1 was linearized by digestion with the restriction enzyme PacI and then transfected into T-REx-293 cells (which express the Tetracycline repressor) using the CaPO₄ method (InVitrogen kit). Ten days later, 10 plaques were picked and grown in T-REx-293 cells in 35-mm plates. PCR analysis of the adenoviral DNA indicated that the virus were positive for HPV16L1.

Evaluation of Large Scale Adenovirus Ad5-TO-HPV16L1

A selected clone was grown into large quantities through multiple rounds of amplification in T-REx-293 cells. Viral DNA was extracted and confirmed by PCR and restriction enzyme analysis. Expression of HPV16L1 was verified by immunoblot analysis of 293 cells infected with the recombinant adenovirus. (Expression from the CMV-TO promoter is depressed in 293 cells, which do not express the Tetracycline repressor).

Construction of Ad5-TO-HPV16E2.

The construction of V1Jns-HPV16E2 containing the codon-optimized HPV16E2 coding sequence was described previously (WO 01/14416A2). The coding sequence for HPV16E2 was excised from V1Jns-HPV16E2 by digestion with BglII and the fragment was made flushended. The aforementioned shuttle vector pA1-TO-HPV16L1 was digested with BamHI plus EcoRV to remove the HPV16L1 coding sequence. The resulting vector fragment (pA1-TO) was then made flush-ended by treatment with Klenow DNA polymerase and ligated with the HPV16E2 DNA fragment, yielding the shuttle vector pA1-TO-HPV16E2. This latter shuttle vector was digested with restriction enzymes SgrAI and BstZ17I and then co-transformed into E. coli strain BJ5183 with linearized (ClaI-digested) adenoviral backbone plasmid pAdE1-E3-. The resulting transformants were screened and recombinant Ad5-TO-HPV16E2 virus was rescued and expanded in T-REx-293 cells as described above. Expression of HPV16E2 was verified by immunoblot analysis of 293 cells infected with the recombinant adenovirus.

Construction of Ad5-COPVE1: The coding sequence for COPV E1 was excised from V1Jns-COPV-E1 by digestion with EcoRV and ligated with the aforementioned shuttle EcoRV-BamHI(flushended) pA1-TO vector fragment., yielding the shuttle vector pA1-TO-COPV-E1. This shuttle vector was then digested with SgrAI plus BstZ17I and co-transfected into E. coli strain BJ5183 with linearized (ClaI-digested) adenovirus vector backbone pAdE1-E3. The resulting transformants were screened and recombinant adenovirus, Ad5-COPVE1, was then rescued and amplified in T-Rex-293 cells as described above. Expression of COPVE1 was verified by immunoblot analysis of 293 cells infected with the recombinant adenovirus.

Construction of Ad5-COPVE2:: The coding sequence for COPV E2 was excised from V1Jns-COPV-E2 by digestion with PmlI and ligated with the aforementioned EcoRV-BamHI(flushended) pA1-TO vector fragment, yielding the shuttle vector pA1-TO-COPV-E2. This shuttle vector was then digested with SspI plus BstZ17I and co-transformed into E. coli strain BJ5183 with linearized (ClaI-digested) adenovirus vector backbone pAdE1-E3—DNA as described above. Eight single colonies were picked from the resulting transformation plate and inoculated into 2-ml of Terrific Broth with ampicillin (50 mcg/ml) and then grown for 8 hours at 37° C. Cells were harvested and small-scale plasmid DNA preparations were made (pAd-TO-COPV-E2 isolates). The plasmid DNAs for pAd-TO-COPV-E2 clones #1, 3, 5 and 7 were then transformed into E. coli STBL2 competent cells. Two colonies for each original DNA (colonies 1-1, 1-2, 3-1, 3-2, 5-1, 5-2, 7-1 and 7-2) were picked and grown separately in LB with ampicillin (50 mcg/ml) overnight at 37° C. Large-scale plasmid DNA preparations were then made for pAd-TO-COPV-E2 isolates #7-1 and #7-2. Both purified DNAs were digested with HindIII and XhoI to confirm that they had the correct structure. Both pAd-TO-COPV-E2 isolates #7-1 and #7-2 were digested with PacI and transfected into T-REx-293 cells using GTS Geneporter transfection reagent. Six days later, several plaques were picked and grown in T-REx-293 cells in 35 mm plates. Based on PCR analysis of the adenoviral DNA, clone #7.1B of Ad-TO-COPV-E2 was selected for further evaluation. This isolate was grown into large quantities through multiple rounds of amplification in T-REx-293 cells. The virus was then purified by banding on CsCl equilibrium density gradients. This virus preparation was designated Ad5-COPVE2, ID#7.1 p7. Viral DNA was purified and the structure was confirmed by digestion with the restriction enzymes HindIII and XhoI. Expression of COPV E2 was verified by immunoblot analysis of 293 cells infected with the recombinant Ad5-COPVE2 adenovirus.

Construction of Ad5-COPVE4 and Ad5-COPVE7: The coding sequences for COPV E4 and COPV E7 (C24G, E26G double mutant) were excised from V1Jns-COPV-E4 and V1Jns-COPV-E7, respectively, by digestion with PmlI. The gene fragments were ligated with the aforementioned EcoRV-BamHI(flushended) pA1-TO vector fragment, yielding the shuttle vectors pA1-TO-COPV-E4 and pA1-TO-COPV-E7, respectively. The subsequent steps of recombination with the pAdE1-E3—vector backbone and the rescue and amplification of the resulting recombinant Ad5-COPVE4 and Ad5-COPVE7 viruses in T-REx-293 cells were as described above. Expression of COPV E4 and COPV E7 was verified by immunoblot analyses of 293 cells infected with the corresponding recombinant adenovirus.

### EXAMPLE 5

Generation of HPV-Specific Cellular Immune Responses in Mice by Immunization with Ad-TO-HPV16E2 or Ad-TO-HPV16L1

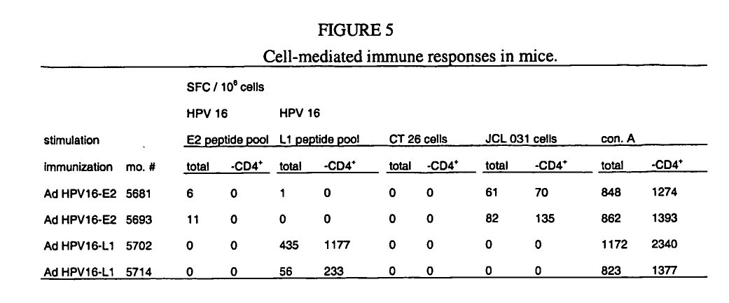

Groups of female BALB/c mice were immunized by intramuscular injection with 10⁹ virus particles (vp) Ad-TO-HPV16E2 or with 10⁹ vp Ad-TO-HPV16L1 (control) at day 0 and day 21. On day 34, two mice from each immunization group were randomly chosen, sacrificed, and ELISPOT analysis was performed on splenocytes. The results are shown in FIG. 5. Animals immunized with Ad-TO-HPV16E2 had developed only HPV 16 E2-specific responses, while the Ad-TO-HPV16L1-immunized animals developed only HPV 16 L1-specific responses.

### EXAMPLE 6

IFN-γ ELISpot Assay

Mouse splenocytes were prepared from freshly macerated spleens. Depletion of CD4+ cells was achieved by magnetic bead separation using Dynabeads CD4 (L3T4) (Dynal, Oslo). Briefly, 96-well polyvinylidine difluoride (PVDF)-backed plates (MAIP NOB 10; Millipore, Bedford, Mass.) were coated with 10 μg anti-murine rIFN-γ (BD PharMingen) per well in 100 μl of PBS at 4° C. for 16-20 hours. Plates were washed three times with PBS, and then blocked with RPMI-1640 medium containing 10% heat-inactivated FBS. Cells were cultured at 5×10⁵ per well in 0.1 mL of medium for restimulation with pools of 20mer peptides comprising the entire amino acid sequence of HPV16 E2, or L1 or matching DMSO concentration in media as a negative control.

Alternatively, cells were co-cultured with 10⁴ CT26 cells, a fully-transformed, tumorigenic syngeneic line, or with 10⁴ JCL031 cells, a clonal isolate derived from CT26 cells that had been transformed to express HPV 16 E2 protein. After 20-24 hr incubation at 37° C., the plates were washed 6 times with PBS containing 0.005% Tween 20. Plates were then incubated with 1 μg biotinylated anti-murine rIFN-γ (BD PharMingen) per well in 50 μl of PBS-Tween+5% FCS at 4° C. for 16-20 hours. The plates were washed 6 times with PBS-Tween before the addition of 100 μl per well of Streptavidin-AP conjugate (BD PharMingen), diluted 1:2000 in PBS-Tween+5% FCS. After 3 washes with PBS-Tween and 3 washes with PBS, spots were developed with one-step NBT/BCIP reagent (Pierce, Rockford, Ill.). Spots were counted using an automated detection system.

### EXAMPLE 7

Protection of Mice from an HPV E2 Tumor Challenge by Immunization with Ad-TO-HPV16E2

Groups of BALB/c mice were immunized by intramuscular injection with 10⁹ vp Ad-TO-HPV16E2 or with 10⁹ vp Ad-TO-HPV16L1 (control) at day 0 and day 21. On day 43, each group of 18 mice were challenged by s.c. inoculation with 7.5×10⁵ JCL031 cells, a fully-transformed tumorigenic, isogenic cell line that expresses HPV16 E2 derived from the CT26 cell line.

Briefly, the plasmid, pBJ-16 E2, which induces E2 protein expression in transiently-transfected A293 or CT26 cells, was transfected into CT26 cells using Lipofectamine (Gibco BRL, Gaithersburg, Md.). CT26 cells, a fully-transformed line derived from a BALB/c mouse colon carcinoma, have been widely used to present model tumor antigens. (Brattain et al., 1980 Cancer Research 40:2142-2146; Fearon, E. et al., 1988 Cancer Research, 48:2975-2980; both of which are incorporated by reference). After two to three weeks growth in selective medium containing 400 μg/mL G418, well-isolated colonies of cells were recovered using cloning rings and transferred to 48-well plates. One clone was positive for E2 expression by immunoblot analysis and was subjected to two further rounds of cloning by limiting dilution. One G418 resistant, E2-positive clonal isolate was used to established the cell line JCL-031.

Animals were monitored for tumor outgrowth for four weeks. The results are shown in FIG. 2. Animals immunized with the Ad-TO-HPV16E2 virus were well-protected from tumor out-growth; 17 of 18 remained tumor-free during the observation period. In the control group, 16 of 18 mice developed tumors.

### EXAMPLE 8

Generation of HPV16-Specific Cellular Immune Responses in Rhesus Macaques by Immunization with Ad5 HPV-16 Constructs

Cohorts of 3 or 4 Rhesus macaques were vaccinated intramuscularly at weeks 0 and 24 with 10¹¹ Ad5-TO-HPV16L1, Ad5 HPV16-E1, or Ad5 HPV16-L2 virus particles. PBMC samples were collected at selected time points and assayed for antigen-specific IFN-γ secretion following overnight stimulation with HPV16 L1, E1, or E2 20mer peptide pools via ELISpot assay.

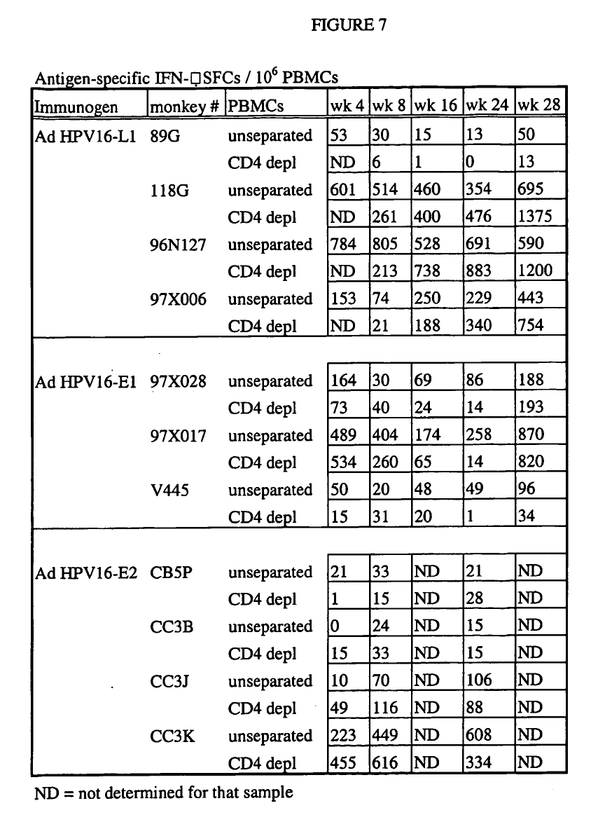

The results shown in FIG. 7 demonstrate a strong cellular immune response to HPV16 L1, E1, and E2 following a single dose of the Ad5 HPV16 constructs. These data also demonstrate that the cellular responses can be boosted by vaccination with a second dose of the Ad5 HPV16 constructs.

### EXAMPLE 9

IFN-γ ELISpot Assay

Rhesus macaque Peripheral Mononuclear Cells (PBMCs) were isolated from freshly drawn heparinized blood by Ficoll density gradient centrifugation. Depletion of CD4+ cells was achieved by magnetic bead separation using Dynabeads M-450 CD4 (Dynal, Oslo).

Briefly, 96-well polyvinylidine difluoride (PVDF)-backed plates (MAIP NOB 10; Millipore, Bedford, Mass.) were coated with 10 μg anti-human rIFNγ (R&D Systems Minneapolis, Minn.) per well in 100 μl of PBS at 4° C. for 16-20 hours. Plates were washed three times with PBS, and then blocked with RPMI-1640 medium containing 10% heat-inactivated FBS. Cells were cultured at 5×10⁵ per well in 0.1 mL of medium for restimulation with pools of 20mer peptides comprising the entire amino acid sequence of HPV16E1, E2, or L1 or matching DMSO concentration in media as a negative control. After 20-24 hr incubation at 37° C., the plates were washed 6 times with PBS containing 0.005% Tween 20. Plates were then incubated with 1 μg biotinylated anti-human rIFN-γ (R&D Systems) per well in 50 μl of PBS-Tween+5% FCS at 4° C. for 16-20 hours. The plates were washed 6 times with PBS-Tween before the addition 100 μl per well of Streptavidin-AP conjugate (BD Pharmingen), diluted 1:2000 in PBS-Tween+5% FCS. After 3 washes with PBS-Tween and 3 washes with PBS, spots were developed with one-step NBT/BCIP reagent (Pierce). Spots were counted using a stereomicroscope.

### EXAMPLE 10

Protection of Beagle Dogs from Canine Oral Papillomas Using Recombinant Adenovirus Constructs Expressing COPV E Proteins

Groups of 4-10 beagle dogs were immunized twice s.c. with 10¹¹ vp per dose at Day 0 and Day 30 with recombinant adenoviruses expressing COPV E proteins or HPV16 L1 as a negative control. Dogs were challenged by scarification at Day 60 at 10 sites of the buccal mucosa. Dogs were monitored weekly for formation of warts at the challenged sites for 16 weeks.

Three experiments were performed: In the first experiment 6 dogs per group were immunized with adenovirus constructs expressing E1+E2, or E4+E7, or E1+E2+E4+E7 and 6 dogs were immunized with an adenovirus control expressing HPV16 L1 (4 groups total). In the second experiment, 5 dogs per group were immunized with recombinant adenoviruses expressing E1+E2, or E1 alone, or E2 alone, and 4 dogs were immunized with control. In the third experiment, 4 dogs per group were immunized with recombinant adenoviruses expressing E1 or E2 alone, or the control vaccine.

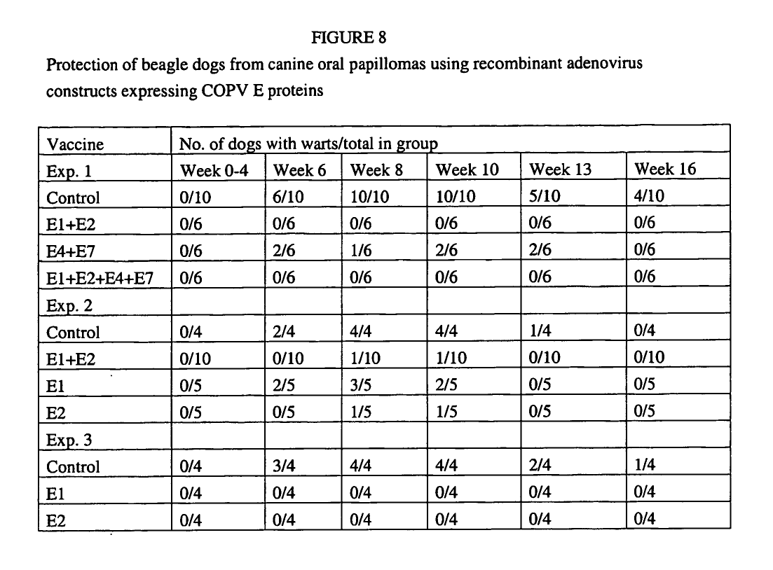

The immunization with COPV E2+E1 adenoviruses almost completely abolished wart formation and greatly reduced the persistence of warts, which appeared. The COPV E2 construct by itself was just as efficacious as the E1+E2 constructs, while the E1 construct by itself initially appeared not to be as potent in reducing disease (Exp. 2) but in a repeat study (Exp. 3) was just as efficacious as the E1+E2 constructs. Also the E4+E7 recombinant adenoviruses were not as potent as the E2 or E1+E2 adenoviruses. Results are shown in FIG. 8.

1. A method of preventing or treating a disease caused by a papillomavirus comprising administering to a mammal a vaccine vector comprising a papillomavirus E gene.

2. A method according to claim 1 wherein the mammal is human.

3. A method according to claim 1 wherein the vector is an adenovirus vector or a plasmid vector, and the genes are from a human papillomavirus (HPV) serotype which is associated with a human disease state.

4. A method according to claim 1 wherein the protein is selected from the group consisting of: E1, E2, E4, E5, E6 and E7 proteins, mutants, and combinations thereof.

5. A method according to claim 4 wherein the protein is E1 or E2 protein.

6. A method according to claim 5 wherein the polynucleotide encoding the E protein is codon-optimized for expression in the recipient's cells.

7. A method according to claim 1 wherein the vector is an adenoviral vector comprising an adenoviral genome with a deletion in the adenovirus E1 region, and an insert in the adenovirus E1 region, wherein the insert comprises an expression cassette comprising:
a) a polynucleotide encoding a papillomavirus protein selected from the group consisting of E1, E2, E4, E5, E6, E7, and combinations thereof, or mutant forms thereof, wherein the polynucleotide is codon-optimized for expression in a human host cell; and
b) a promoter operably linked to the polynucleotide.

8. A method according to claim 1 wherein the vector is a shuttle plasmid vector comprising a plasmid portion and an adenoviral portion, the adenoviral portion comprising: an adenoviral genome with a deletion in the adenovirus E1 region, and an insert in the adenovirus E1 region, wherein the insert comprises an expression cassette comprising:
a) a polynucleotide encoding an E protein selected from the group consisting of—E1, E2, E4, E5, E6, E7, and combinations thereof, or mutant forms thereof, wherein the polynucleotide is codon-optimized for expression in a mammalian host cell; and
b) a promoter operably linked to the polynucleotide.

9. A method according to claim 1 wherein the vector is a plasmid vaccine vector, which comprises a plasmid portion and an expressible cassette comprising
a) a polynucleotide encoding an E protein selected from the group consisting of E1, E2, E4, E5, E6, E7 and combinations thereof, or mutant forms thereof, wherein the polynucleotide is codon-optimized for expression in a mammalian host cell; and
b) a promoter operably linked to the polynucleotide.

10-18. (canceled)

19. A synthetic polynucleotide comprising a sequence encoding a canine papillomavirus (COPV) protein, or a mutated form of a COPV protein, the polynucleotide sequence comprising codons optimized for expression in a human host.

20. A polynucleotide according to claim 19 wherein the protein is selected from the group consisting of; E1, E2, E3, E4, E5, E6, E7, mutants thereof and combinations thereof.

21. A polynucleotide according to claim 20 which is selected from the group consisting of E1, E2, E4+E7, and E1+E2+E4+E7.

22. A polynucleotide according to claim 19 which is DNA.

23. A polynucleotide according to claim 22 which is selected from the group consisting of SEQ.ID.NO. 1, SEQ.ID.NO. 2, SEQ.ID.NO. 3, SEQ.ID.NO. 4, and combinations thereof.

24. An adenovirus vaccine vector comprising and adenoviral genome with a deletion in the E1 region, and an insert in the E1 region, wherein the insert comprises an expression cassette comprising:
a) a polynucleotide encoding a COPV protein selected from the group consisting of E1, E2, E3, E4, E5, E6, E7, mutants thereof, and combinations thereof, wherein the polynucleotide is codon optimized for expression in a host cell; and
b) a promoter operably linked to the polynucleotide.

25. An adenovirus vector according to claim 24 which is an Ad 5 vector.

26. A vaccine plasmid comprising a plasmid portion and an expression cassette portion, the expression cassette portion comprising:
a) a polynucleotide encoding a COPV protein selected from the group consisting of E1, E2, E3, E4, E5, E6, E7, mutants thereof, and combinations thereof, wherein the polynucleotide is codon optimized for expression in a host cell; and
b) a promoter operably linked to the polynucleotide.

27. method of protecting a mammal from or treating a mammal with a papillomavirus disease comprising:
a) introducing into the mammal a first vector comprising:
i) a polynucleotide encoding an HPV or COPV protein selected from the group consisting of E1, E2, E3, E4, E5, E6, E7, mutants thereof, and combinations thereof, wherein the polynucleotide is codon optimized for expression in a host cell; and
ii) a promoter operably linked to the polynucleotide;
b) allowing a predetermined amount o time to pass; and
c) introducing into the mammal a second vector comprising:
i) a polynucleotide encoding an HPV or COPV protein selected from the group consisting of E1, E2, E3, E4, E5, E6, E7, mutants thereof, and combinations thereof, wherein the polynucleotide is codon optimized for expression in a host cell; and
ii) a promoter operably linked to the polynucleotide.

28. A method according to claim 27 wherein the first vector is a plasmid and the second vector is an adenovirus vector.

29. A method according to claim 28 wherein the first vector is an adenovirus vector and the second vector is a plasmid.

30-32. (canceled)


SEQUENCE LISTING (4 sequences)

SEQ ID NO: 1 - DNA, 1794 bp, Artificial Sequence
  condon-optimized COPV El gene
atggccgctcgcaagggcaccgacagcgagaccgaggacggcgggtgggtgctgatcgaggccgactgcagcgaggtggacagcgccgacgagaccagcgagaacgccagcaacgtgagcgacctggtggacaacgccagcatcgccgagacccagggcctgagcctgcagctgttccagcaacaggagctgaccgagtgcgaagagcagctgcaacagctgaagcgcaagttcgtgcagagccctcagagccgggacctgtgctctctgagccctcagctggccagcatcagcctgactccccgcaccagcaagaaggtgaagaaacagctgttcgccaccgacagcgggatccagagctccaacgaggccgacgacagcctcgagggccagcgccaggtggagcccctgcccggcagggaggagaacggcgccgacgccctgttcaaggtgcgcgacaagcgcgccttcctgtacagcaagttcaagagcagcttcggcatcagcttcaccgacctgacacgcgtgtacaacagcgacaagacctgcagcagcgactgggtggtgtgcctgtaccatgtgagcgacgaccgccgcgaggccggcaagaccctgctgcaggaccactgcgagtacttcttcctgcacagcatgggcttctgcaccctgctcctgctctgcctgttcgtgcccaagtgccgcaacaccctgttcaagctgtgccgcagcctgttccacatcagcaacgtgcagatgctggccgaccctcccaagacccgcagccccgctgtggccctgtactggtacaagaagggcttcgccagcggtaccttcacccacggcgagctgcccagctggatcgcccagcagaccctgatcacccatcacctggccgccgagaagaccttcgacctgagcgagatggtgcagtgggcgtacgacaacgacctgaaggacgagagcgagatcgcctacaagtacgccgctctggccgagaccgacgagaacgccctggccttcctgaagagcaacaatcagcccaagcacgtgaaggactgcgccaccatgtgccgctactacaagaaggccgagatgaagcgcctgagcatgagccagtggatcgacgagcgctgcaaggccaccgacgacggtcccggggattggaaggaggtggtgaagttcctgcgccaccagggcatcgaattcatcctgttcctggccgacttcaagcgcttcctgcgcggccgccctaagaagaactgcctggtgttctggggccctcccaacaccggcaagagcatgttctgcatgagcctgctgagcttcctgcacggcgtggtgatcagctacgtgaacagcaagagccacttctggctgcagcccctgaccgagggcaagatgggcctcctggacgatgccacccgcccctgctggctgtacatcgatacctacctgcgcaacgccctggacggcaacaccttcagcgtcgactgcaagcacaaggctcccctgcagctgaagtgccctcccctgctcatcaccaccaacgtgaacgtctgcggcgacgagaagttcaagtacctgcgcagccgctgcagcttcttccacttccctcaggagtttcccctggacgacaacggcaatcccggcttccagctgaacgaccagagctgggccagctttttcaagcgcttctggaagcacctggacctgagcgaccccgaggacggcgaggacggcgagacccagcgcggcctgcgcctgaccgctcgcggcaccaccgagagcgtgtaa

SEQ ID NO: 2 - DNA, 1158 bp, Artificial Sequence
  condon-optimized COPV EZ gene
atggagaagctgagcgaggccctggacctgctgcaggaggagctgctgagcctgtacgagcagaacagccagagcctggccgaccagagccgccactggagcctgctgcgcaaggagcaggtgctgctgtactacgcccgcggcaagggcatcatgcgcatcggcatgcagcccgtgcctccccagagcgtgagccaggccaaggccaagcaggccatcgagcagagcctgtacatcgacagcctgctgcacagcaagtacgccaacgagccatggaccctgtgcgacaccagccgcgagcgcctggtggccgagcctgcctacaccttcaagaagggcggcaagcagatcgacgtgcgctacggcgacagcgaggagaacatcgtgcgctacgtgctgtggctggacatctactaccaggacgaattcgacacctgggagaaggcccacggcaagctggaccacaagggcctgagctacatgcacggcacccagcaggtgtactacgtcgacttcgaggaggaggccaacaagtacagcgagaccggcaagtacgagatcctgaaccagcccaccaccatccctaccaccagcgccgctggcaccagcggccccgagctgcctggccacagcgcctcggggtccggtgcctgttcccttacccccaggaaagggccgtcacggcggcctggacggaggtcgtcgcggttccccagaaggtcaggaggacgaggaagactcggacgaggaggaagcggagaattacccccccagccgcagccgtcctcgtcgtggtcgccgccgtctccacaacaagtgggatcaaaacatcaactacgaaccaccagcagcgccggcggccgcctgggccgcctgctgcaggaggcctacgaccctcccgtgctggtgctggccggtgaccccaacagcctgaagtgcatccgctaccgcctgagccacaagcaccgcggcctgtacctgggcgccagcaccacctggaagtggaccagcggcggcgacggcgccagcaagcacgaccgcggcagcgcccggatgctgctggccttcctgagcgaccagcagcgcgaggacttcatggaccgcgtgaccttccccaagagcgtgcgcgtgttccgcggcggcctggacgagctgtaa

SEQ ID NO: 3 - DNA, 357 bp, Artificial Sequence
  condon-optimized COPV E4 gene
atgcgcttcaccaaccccctgctgttcccccctcccgtgcctcccgagcctcccgaccgcaacagcccggtgacccctccacgcggacctgtgcctgtgccactgccgcctggcaagggcaggcacggtggactggacggtggccgccgcggcagccctgagggccaggaggacgaggaggacagcgacgaggaagaggccgagaactaccctcccagccgcagccgccctcgccgcggccgccgccgcctgcacaacaagtgggaccagaacatcaactacgagcctcccgccgcccccgaggacgactgggaggacttctgcaagaagctgaccatcccccagttcctgttctaa

SEQ ID NO: 4 - DNA, 294 bp, Artificial Sequence
  condon-optimized mutant COPV E7 (C24G, E26G) gene
atgatcggccagtgcgccaccctgctggacatcgtgctgaccgagcagcccgagcccatcgacctgcagggatacggccagctgcccagcagcgacgaggaggaggaagaggaggagcccaccgagaagaacgtgtaccgcatcgaggccgcctgcggcttctgcggcaagggcgtgcgcttcttctgcctgagccagaaggaggacctgcgcgtgctgcaggtgaccctgctgagcctgagcctggtgtgcaccacctgcgtgcagaccgccaagctggaccatggcggctaa